SEQUENCE LISTING (8 sequences)

SEQ ID NO: 1 - DNA, 110 bp, Homo sapiens
  source 1..110: mol_type = genomic DNA
ggtttttgattcggattacggttttttttttgaggtgtttttgagtcgtagtttttttttgagtgttttttttttgtagggtttttgtacgttggggttttttaggattg

SEQ ID NO: 2 - DNA, 21 bp, synthetic construct
  source 1..21: mol_type = other DNA
ggtttttgattcggattacgg

SEQ ID NO: 3 - DNA, 23 bp, synthetic construct
  source 1..23: mol_type = other DNA
caatcctaaaaaaccccaacgta

SEQ ID NO: 4 - DNA, 133 bp, Homo sapiens
  source 1..133: mol_type = genomic DNA
tggtgatggaggaggtttagtaagttttttggattgtgaatttgtgtttgttattgtgtgttgggtggtggttattttttttattaggttgtggtttttgtaatttttaagggaggagtaggttttattggtt

SEQ ID NO: 5 - DNA, 25 bp, Synthetic construct
  source 1..25: mol_type = other DNA
tggtgatggaggaggtttagtaagt

SEQ ID NO: 6 - DNA, 27 bp, synthetic construct
  source 1..27: mol_type = other DNA
aaccaataaaacctactcctcccttaa

SEQ ID NO: 7 - DNA, 25 bp, synthetic construct
  source 1..25: mol_type = other DNA
tttgaggtgtttttgagtcgtagtt

SEQ ID NO: 8 - DNA, 30 bp, synthetic construct
  source 1..30: mol_type = other DNA
accaccacccaacacacaataacaaacaca

(19) United States
(12) Patent Application Publication
(10) Pub. No.: US 2026/0125759 A1
(43) Pub. Date: May 7, 2026

(54) BIOMARKERS ASSOCIATED WITH AGING

(71) Applicant: EG BIOMED CO., LTD., TAIPEI CITY (TW)

(72) Inventors: Hsieh-Tsung SHEN, TAIPEI CITY (TW); Ruo-Kai LIN, TAIPEI CITY (TW)

(21) Appl. No.: 19/376,147

(22) Filed: Oct. 31, 2025

Related U.S. Application Data
(60) Provisional application No. 63/715,284, filed on Nov. 1, 2024.

(51) Int. Cl. C12Q 1/6883

(52) U.S. Cl. CPC C12Q 1/6883; C12Q 2600/154

(57) ABSTRACT

The present invention discloses a novel epigenetic biomarker for detection of aging. Particularly, the present disclosure assesses methylation of the epigenetic biomarkers to determine the aging status of a subject. The present disclosure also discloses primers and probes used herein.

### CROSS-REFERENCE TO RELATED APPLICATIONS

This application claims benefit of and priority to U.S. Provisional Patent Application No. 63/715,284, filed 1 Nov. 2024, the contents of which is incorporated by reference in its entirety.

### SEQUENCE LISTING

The instant application contains a Sequence Listing which is submitted electronically in.xml format and is hereby incorporated by reference in its entirety. The .xml copy, created on Oct. 31, 2025, is named “US16327_SequenceListing.xml” and is 8,362 bytes in size.

### FIELD OF THE INVENTION

The present disclosure relates to the field of gene biomarkers. Particularly, the present disclosure assesses methylation of the gene biomarkers to determine the aging status of a subject.

### BACKGROUND OF THE INVENTION

Aging refers to the gradual decline of essential physiological functions needed for survival and reproduction as time passes. It is a continuous, natural process that starts in early adulthood and impacts every member of a species. This process results from the accumulation of various molecular and cellular damages over time, causing a slow reduction in both physical and cognitive abilities, an increased vulnerability to diseases, and eventually, death.

DNA methylation at the fifth carbon position of cytosine (5mC) within CpG dinucleotides is a stable epigenetic modification that plays a crucial role in mammalian development, cell differentiation, and the preservation of cellular identity by regulating gene expression. Aberrant patterns of DNA methylation have been linked to various diseases and health conditions.

However, there is still a need for novel epigenetic biomarkers to assess the aging status of a subject.

### SUMMARY OF THE INVENTION

The present invention discloses a novel epigenetic biomarker for detection of aging. Particularly, the present disclosure assesses methylation of the epigenetic biomarkers to determine the aging status of a subject. The present disclosure also discloses primers and probes used herein.

### Detection of Aging

In one embodiment, the present disclosure provides a method for detecting an aging status of a subject, wherein the method comprises:
(a) extracting (i) cell-free DNA (cfDNA) from a biological sample of the subject or (ii) genomic DNA from a biological sample of the subject, wherein the cfDNA or the genomic DNA comprises a DENND2B DNA and a reference gene DNA;
(b) assaying a methylation level of one or more CpG site of the DENND2B DNA from the cfDNA or the genomic DNA;
(c) measuring a relative nucleic acid quantity of the reference gene DNA from the cfDNA or the genomic DNA; and
(d) determining an aging status of the subject based on a combination of the methylation level and the relative nucleic acid quantity.

In some embodiments, a pair of DENND2B methylation-specific primers used to assay the methylation level of one or more CpG site of the DENND2B DNA comprises sequences having at least 85% identity to SEQ ID NO: 2 and SEQ ID NO: 3. In some embodiments, the pair of DENND2B methylation-specific primers comprises sequences having about 85%, about 86%, about 87%, about 88%, about 89%, about 90%, about 91%, about 92%, about 93%, about 94%, about 95%, about 96%, about 97%, about 98%, about 99%, or about 100% identity to SEQ ID NO: 2 and SEQ ID NO: 3. In one embodiment, the pair of DENND2B methylation-specific primers comprises the sequences of SEQ ID NO: 2 and SEQ ID NO: 3. In another embodiment, the pair of DENND2B methylation-specific primers have the sequences of SEQ ID NO: 2 and SEQ ID NO: 3.

In some embodiments, a DENND2B methylation-specific probe used to assay the methylation level of one or more CpG site of the DENND2B DNA comprises a sequence having at least 85% identity to SEQ ID NO: 7. In some embodiments, the DENND2B methylation-specific probe comprises a sequence having about 85%, about 86%, about 87%, about 88%, about 89%, about 90%, about 91%, about 92%, about 93%, about 94%, about 95%, about 96%, about 97%, about 98%, about 99%, or about 100% identity to SEQ ID NO: 7. In one embodiment, the DENND2B methylation-specific probe comprises the sequence of SEQ ID NO: 7. In another embodiment, the DENND2B methylation-specific probe has the sequence of SEQ ID NO: 7.

In some embodiments, the reference gene DNA comprises a ACTB DNA.

In some embodiments, a pair of primers used to measure the relative nucleic acid quantity of the reference gene DNA comprises sequences having at least 85% identity to SEQ ID NO: 5 and SEQ ID NO: 6. In some embodiments, the pair of primers used to measure the relative nucleic acid quantity comprises sequences having about 85%, about 86%, about 87%, about 88%, about 89%, about 90%, about 91%, about 92%, about 93%, about 94%, about 95%, about 96%, about 97%, about 98%, about 99%, or about 100% identity to SEQ ID NO: 5 and SEQ ID NO: 6. In one embodiment, the pair of primers used to measure the relative nucleic acid quantity comprises the sequences of SEQ ID NO: 5 and SEQ ID NO: 6. In another embodiment, the pair of primers used to measure the relative nucleic acid quantity has the sequences of SEQ ID NO: 5 and SEQ ID NO: 6.

In some embodiments, a probe used to measure the relative nucleic acid quantity of the reference gene DNA comprises a sequence having at least 85% identity to SEQ ID NO: 8. In some embodiments, the probe used to measure the relative nucleic acid quantity of the reference gene DNA comprises a sequence having about 85%, about 86%, about 87%, about 88%, about 89%, about 90%, about 91%, about 92%, about 93%, about 94%, about 95%, about 96%, about 97%, about 98%, about 99%, or about 100% identity to identity to SEQ ID NO: 8. In one embodiment, the probe used to measure the relative nucleic acid quantity of the reference gene DNA comprises the sequence of SEQ ID NO: 8. In another embodiment, the probe used to measure the relative nucleic acid quantity of the reference gene DNA has the sequence of SEQ ID NO: 8.

In some embodiments, the step of determining the aging status comprises calculating an aging index by binomial regression based on the methylation level and the relative nucleic acid quantity.

In some embodiments, the step of assaying the methylation level of one or more CpG site of the DENND2B DNA is performed using methylation specific PCR (MSP), quantitative methylation-specific PCR (QMSP), bisulfite conversion, bisulfite sequencing (BS), pyrosequencing, microarrays, or methylation DNA immunoprecipitation PCR (MeDIP-PCR).

In some embodiments, the biological sample described herein comprises a liquid biopsy. In one embodiment, the biological sample comprises plasma, serum, or a combination thereof.

In some embodiments, the present disclosure further provides a kit for detecting an aging status of a subject. The kit comprises a pair of primers having the sequences of SEQ ID NOs: 2 and 3 and/or a probe having the sequence of SEQ ID NO: 7 for assaying methylation level of a DENND2B DNA from a biological sample of a subject.

In some embodiments, the kit further comprises a pair of primers having the sequences of SEQ ID NOs: 5 and 6 and/or a probe having the sequence of SEQ ID NO: 8 for measuring a relative nucleic acid quantity of a reference gene DNA from the biological sample. In certain embodiments, the reference gene DNA comprises a ACTB DNA.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1A shows the scatter plot for an aging molecular marker in the female population.

FIG. 1B shows the scatter plot for an aging molecular marker in the male population.

FIG. 2 shows the distribution of an aging molecular marker across different ages.

### DETAILED DESCRIPTION OF THE INVENTION

It is understood that this invention is not limited to the particular materials and methods described herein. It is also to be understood that the terminology used herein is for the purpose of describing particular embodiments and is not intended to limit the scope of the present invention, which will be limited only by the appended claims.

It must be noted that, as used in this specification and the appended claims, the singular forms “a,” “an,” and “the” include plural referents unless the content clearly dictates otherwise. Thus, for example, reference to “a biomarker” includes a mixture of two or more biomarkers, and the like.

The term “biological sample” refers to a sample of tissue, cells, or fluid isolated from a subject, including but not limited to, for example, blood, buffy coat, plasma, serum, blood cells (e.g., peripheral blood mononucleated cells (PBMCS), band cells, neutrophils, metamyelocytes, monocytes, or T cells), fecal matter (such as stool), urine, bone marrow, bile, spinal fluid, lymph fluid, samples of the skin, external secretions of the skin, respiratory, intestinal, and genitourinary tracts, tears, saliva, milk, organs, biopsies and also samples of in vitro cell culture constituents, including, but not limited to, conditioned media resulting from the growth of cells and tissues in culture medium, e.g., recombinant cells, and cell components.

The term “CpG site” as used herein refers to stretches of DNA in a genome that are rich in GC relative to the rest of the genome. Typically, the GC content is 50% or greater in these regions, which extend over hundreds of base pairs and sometimes thousands. Often these regions mark the 5′ ends of genes.

As used herein, the terms “detect”, “detecting” or “detection” may describe either the general act of discovering or discerning or the specific observation of a detectably labeled composition.

The term “gene” refers to a nucleic acid (e.g., DNA) sequence that comprises coding sequences necessary for the production of a polypeptide, precursor, or RNA (e.g., non-coding RNAs such as ribosomal RNA, transfer RNA, splicosomal RNA, microRNA). A polypeptide or non-coding RNA can be encoded by a full-length coding sequence or by any portion of the coding sequence so long as the desired activity or functional properties (e.g., enzymatic activity, ligand binding, signal transduction, immunogenicity, etc.) of the full-length or fragment polypeptide are retained. Accordingly, a gene can include or exclude promoter sequences, terminators, translational regulatory sequences such as ribosome binding sites and internal ribosome entry sites, enhancers, silencers, insulators, boundary elements, replication origins, matrix attachment sites and locus control regions. The term also encompasses the coding region of a structural gene and the sequences located adjacent to the coding region on both the 5′ and 3′ ends for a distance of about 1 kb or more on either end such that the gene corresponds in length to the full-length mRNA. The term “gene” further includes both cDNA and genomic forms of a gene.

The term “reference genes” described herein refers to genes that are consistently and stably expressed at relatively constant levels across different tissues, cell types, developmental stages, and experimental conditions. Reference genes can be used as controls or normalization standards in biological assays such as quantitative PCR (qPCR), western blotting, or RNA sequencing. This normalization helps ensure that observed changes in target gene expression are due to biological differences rather than technical variability.

As used herein, the term “identity” refers to a first sequence which shares a degree of sequence identity with a second sequence, but whose sequence is not identical to that of the second sequence. For example, a polynucleotide including the wild-type sequence of a mutant gene is homologous and non-identical to the sequence of the mutant gene. In some embodiments, the degree of identity between the two sequences is sufficient to allow homologous recombination therebetween, under appropriate stringent conditions.

Techniques for determining nucleic acid and amino acid sequence identity include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100.

In some embodiments, the degree of sequence similarity between polynucleotides can be determined by hybridization of polynucleotides under conditions that allow formation of stable duplexes between homologous regions, followed by digestion with single-stranded-specific nuclease(s), and size determination of the digested fragments. Two nucleic acid, or two polypeptide sequences are substantially homologous to each other when the sequences exhibit at least about 70%-75%, preferably 80%-82%, more preferably 85%-90%, even more preferably 92%, still more preferably 95%, and most preferably 98% sequence identity over a defined length of the molecules, as determined using the methods above. As used herein, substantially homologous also refers to sequences showing complete identity to a specified DNA or polypeptide sequence. DNA sequences that are substantially homologous can be identified in a Southern hybridization experiment under, for example, stringent conditions, as defined for that particular system. See, e.g., Sambrook et al., supra; Nucleic Acid Hybridization: A Practical Approach, editors B. D. Hames and S. J. Higgins, (1985) Oxford; Washington, D.C.; IRL Press).

The term “methylation,” as used herein, refers to the presence of a methyl group added by the action of a DNA methyl transferase enzyme to a cytosine base or bases in a region of nucleic acid, e.g., genomic DNA.

The term “methylation state,” “methylation profile,” “methylation pattern,” or “methylation status” of a nucleic acid molecule refers to the presence or absence of one or more methylated nucleotide bases in the nucleic acid molecule. For example, a nucleic acid molecule containing a methylated cytosine is considered methylated (i.e., the methylation status of the nucleic acid molecule is methylated). A nucleic acid molecule that does not contain any methylated nucleotides is considered unmethylated.

The term “hypermethylation” refers to the average methylation status corresponding to an increased presence of methylated nucleotide bases in the nucleic acid molecule at one or a plurality of CpG dinucleotides within a DNA sequence of a test DNA sample, relative to the amount of methylated nucleotide bases in the nucleic acid molecule found at corresponding CpG dinucleotides within a normal control DNA sample.

The term “hypomethylation” refers to the average methylation status corresponding to a decreased presence of methylated nucleotide bases in the nucleic acid molecule at one or a plurality of CpG dinucleotides within a DNA sequence of a test DNA sample, relative to the amount of methylated nucleotide bases in the nucleic acid molecule found at corresponding CpG dinucleotides within a normal control DNA sample.

The term “subject” refers to humans.

The term “target site” or “target sequence” refers to a nucleic acid sequence that defines a portion of a nucleic acid to which a binding molecule will bind, provided sufficient conditions for binding exist.

In many physiological processes, such as aging, gene promoter CpG islands acquire abnormal hypermethylation, which results in transcriptional silencing that can be inherited by daughter cells following cell division. DNA methylation causing silencing in during the aging process typically occurs at multiple CpG sites in the CpG islands that are present in the promoters of protein coding genes. Alterations of DNA methylation have been recognized as an important index of aging. DNA methylation profiling provides valuable insights into a subject's cellular age and aging progression, serving as an important reference for evaluating physiological status. Accordingly, the present disclosure provides a method and kit for detecting an aging status of a subject.

In some embodiments, the methylation status of one or more of the DENND2B DNA from cell-free DNA (cfDNA) or genomic DNA in a biological sample is assayed to detect the aging status of the subject.

DENND2B is a human gene that encodes a protein belonging to the DENN domain-containing family. The DENND2B protein is a guanine nucleotide exchange factor (GEF) for Rab GTPases, activating Rab proteins by facilitating the exchange of GDP for GTP. The DENND2B sequence and its function are known in the art, e.g., as described on website: https://www.genecards.org/cgi-bin/carddisp.pl?gene=DENND2B.

In some embodiments, the methylation involves a cytosine methylation site. In some instances, cytosine methylation comprises 5-methylcytosine (5-mCyt) and 5-hydroxymethylcytosine. In some cases, a cytosine methylation site occurs within a CpG dinucleotide motif. In other cases, a cytosine methylation site occurs within a CHG or CHH motif, where H represents adenine, cytosine or thymine. In some instances, one or more CpG dinucleotide motif or CpG site forms a CpG island, a short DNA sequence rich in CpG dinucleotide. In some instances, CpG islands are typically, but not always, between about 0.2 to about 1 kb in length. In some instances, the methylation comprises CpG island methylation.

In some embodiments, the methylation status of a gene is assayed by methylation specific PCR (MSP), quantitative methylation-specific PCR (QMSP), bisulfite conversion, bisulfite sequencing (BS), pyrosequencing, microarrays, or methylation DNA immunoprecipitation PCR (MeDIP-PCR).

The term “MSP” refers to Methylation-specific PCR. MSP is described by Herman et al. Proc. Natl. Acad. Sci. USA 93:9821-9826, 1996, and by U.S. Pat. No. 5,786,146, each of which are herein incorporated by reference.

Bisulfite conversion of DNA is a method to assess CpG methylation status. 5-methylcytosine is the most frequent covalent base modification in the DNA of eukaryotic cells. However, 5-methylcytosine positions cannot be identified directly by sequencing or hybridization methods, because 5-methylcytosine has the same base pairing behavior as cytosine. Moreover, the epigenetic information carried by 5-methylcytosine is completely lost during, e.g., PCR amplification.

Bisulfite sequencing is a method for analyzing DNA for the presence of 5-methylcytosine is based upon the specific reaction of bisulfite with cytosine whereby, upon subsequent alkaline hydrolysis, cytosine is converted to uracil which corresponds to thymine in its base pairing behavior. However, 5-methylcytosine remains unmodified under the aforementioned conditions. Thus, the original DNA is converted in such a manner that methylcytosine, which originally could not be distinguished from cytosine by its hybridization behavior, can be detected as the only remaining cytosine using molecular biological techniques, for example, by amplification and hybridization, or by sequencing.

In one embodiment of the present disclosure, the sequence of DENND2B selected as a target sequence for assaying the methylation level of DENND2B is:

(SEQ ID NO: 1)
GGTTTTTGATTCGGATTACGGTTTTTTTTTTGAGGTGTTTTTGAG
TCGTAGTTTTTTTTTGAGTGTTTTTTTTTTGTAGGGTTTTTGTAC
GTTGGGGTTTTTTAGGATTG.

In one embodiment of the present disclosure, the sequence of the reference gene (e.g., ACTB) selected as a target sequence for measuring the relative nucleic acid quantity of the reference gene is:

(SEQ ID NO: 4)
TGGTGATGGAGGAGGTTTAGTAAGTTTTTTGGATTGTGAATTTGT
GTTTGTTATTGTGTGTTGGGTGGTGGTTATTTTTTTTATTAGGT
TGTGGTTTTTGTAATTTTTAAGGGAGGAGTAGGTTTTATTGGTT.

In one embodiment of the present disclosure, primer(s) that could amplify a methylated CpG of the genes described herein might be used. The primer(s) includes at least one or more CpG dinucleotide in a region which hybridizes to the methylated CpG of the genes. Specifically, the primer(s) for amplifying a methylated CpG of the genes includes sequence(s) with about 85%, about 86%, about 87%, about 88%, about 89%, about 90%, about 91%, about 92%, about 93%, about 94%, about 95%, about 96%, about 97%, about 98%, about 99%, or about 100% identity to sequence(s) selected from the group consisting of the following sequences.

| SEQ ID | | |
|---|---|---|
| No. | Primer | Sequence |
| 2 | DENND2B_ | GGTTTTTGATTCG |
| | qMSP_F | GATTACGG |
| 3 | DENND2B_ | CAATCCTAAAAAA |
| | qMSP_R | CCCCAACGTA |
| 5 | ACTB-F | TGGTGATGGAGGA |
| | | GGTTTAGTAAGT |
| 6 | ACTB-R | AACCAATAAAACC |
| | | TACTCCTCCCTTAA |

Probe(s) capable of hybridizing with a methylated CpG of the genes described herein might be used. The probe(s) capable of hybridizing with a methylated CpG of the genes comprise at least one or more CpG dinucleotide in a region which hybridizes to the methylated CpG of the genes. Specifically, probe(s) might include sequence(s) with about 85%, about 86%, about 87%, about 88%, about 89%, about 90%, about 91%, about 92%, about 93%, about 94%, about 95%, about 96%, about 97%, about 98%, about 99%, or about 100% identity to sequence(s) selected from the group consisting of the following sequences.

| No. | Probe | Sequence |
|---|---|---|
| 7 | DENND2B qMSP | /56-FAM/TT TGA GGT |
| | probe | G/ZEN/T TTT TGA GTC |
| | | GTA GTT/3IABKFQ/ |
| | | (TT TGA GGT GT TTT |
| | | TGA GTC GTA GTT; SEQ |
| | | ID NO: 7) |
| 8 | ACTB probe | /56-FAM/AC CAC CAC |
| | | C/ZEN/C AAC ACA CAA |
| | | TAA CAA ACA CA/ |
| | | 3IABKFQ/ |
| | | (AC CAC CAC CC AAC |
| | | ACA CAA TAA CAA ACA |
| | | CA; SEQ ID NO: 8) |

* 56-FAM refers to the addition of a 6-carboxyfluorescein (6-FAM) fluorescent dye to 5′ end of an oligonucleotide.
* ZEN refers to a proprietary internal quencher developed by Integrated DNA Technologies (IDT), often used in dual-quenched probes: 5′ fluorophore - internal ZEN quencher - 3′ quencher.
* 3IABKFQ refers to a 3′ Iowa Black® FQ quencher attached at the 3′ end of an oligonucleotide.

In some embodiments, the biological sample comprises a liquid biopsy. In one embodiment, the biological sample comprises plasma, serum, or a combination thereof.

In some embodiments, the hypermethylation status of the epigenetic biomarkers in DNA sequences described herein correlates with an increased risk of aging of a subject compared to a subject without such a hypermethylation status.

In some embodiments, the present disclosure provides a probe having the sequence of SEQ ID NO: 7 or SEQ ID NO: 8. In some other embodiments, the present disclosure provides a primer having the sequence selected from the group consisting of: SEQ ID NOs: 2, 3, 5, and 6.

In some embodiments, the present disclosure provides a kit for detecting an aging status of a subject. The kit comprises a pair of primers having the sequences of SEQ ID NOs: 2 and 3 and/or a probe having the sequence of SEQ ID NO: 7 for assaying methylation level of a DENND2B DNA from a biological sample of a subject.

In some instances, the kit further comprises a pair of primers having the sequences of SEQ ID NOs: 5 and 6 and/or a probe having the sequence of SEQ ID NO: 8 for measuring a relative nucleic acid quantity of a reference gene DNA from the biological sample. In certain instances, the reference gene DNA comprises a ACTB DNA.

In some embodiments, the kits include sodium bisulfite, primers and adapters for whole target genes amplification, and polynucleotides (e.g., detectably-labeled polynucleotides) to quantify the presence of the converted methylated and or the converted unmethylated sequence of at least one cytosine from a DNA region of an epigenetic biomarker described herein.

In some embodiments, the kits include methylation sensing restriction enzymes, primers and adapters for whole target genes amplification, and polynucleotides to quantify the number of copies of at least a portion of a DNA region of an epigenetic marker described herein. In some embodiments, the kits include a methylation binding moiety and one or more polynucleotides to quantify the number of copies of at least a portion of a DNA region of a marker described herein.

The inventions described and claimed herein have many attributes and embodiments including, but not limited to, those set forth or described or referenced in this Detailed Disclosure. It is not intended to be all-inclusive and the inventions described and claimed herein are not limited to or by the features or embodiments identified in this Detailed Disclosure, which is included for purposes of illustration only and not restriction. A person having ordinary skill in the art will readily recognize that many of the components and parameters may be varied or modified to a certain extent or substituted for known equivalents without departing from the scope of the invention. It should be appreciated that such modifications and equivalents are herein incorporated as if individually set forth. The invention also includes all of the steps, features, compositions and compounds referred to or indicated in this specification, individually or collectively, and any and all combinations of any two or more of said steps or features.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents. Reference to any applications, patents and publications in this specification is not, and should not be taken as, an acknowledgment or any form of suggestion that they constitute valid prior art or form part of the common general knowledge in any country in the world.

The specific methods and kits described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” may be replaced with either of the other two terms in the specification. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants. Furthermore, titles, headings, or the like are provided to enhance the reader's comprehension of this document, and should not be read as limiting the scope of the present invention. Any examples of aspects, embodiments or components of the invention referred to herein are to be considered non-limiting.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

Without further elaboration, it is believed that one skilled in the art can utilize the present invention to its fullest extent on the basis of the preceding description. The following examples are, therefore, to be construed as merely illustrative and not a limitation of the scope of the present invention in any way.

Although the present disclosure has been described with an exemplary embodiment, various changes and modifications may be suggested to one skilled in the art. It is intended that the present disclosure encompass such changes and modifications as fall within the scope of the appended claims.

### EXAMPLE

### Example 1: Methylation Levels of Aging Molecular Marker Among Female and Male Populations

The β values obtained from the Illumina EPIC methylation array were analyzed in 80 healthy subjects, divided into four age groups: <30 years, 30-45 years, 45-60 years, and >60 years, with 20 subjects in each group. The methylation levels of the DENND2B marker are presented as β values (beta values). ACTB serves as a reference marker for the relative amount of circulating cell-free nucleic acids in plasma and is represented as Cp values obtained through quantitative real-time PCR (qPCR).

TABLE A
Female Population
Female Population

| | | | | |
|---|---|---|---|---|
| Age | <30 y | 30~45 y | 45~60 y | >60 y |

β value

| | | | | |
|---|---|---|---|---|
| DE-2B | 0.149 | 0.341 | 0.619 | 0.761 |

Cp value

| | | | | |
|---|---|---|---|---|
| ACTB | 33.18 | 33.24 | 32.76 | 32.60 |

TABLE B
Male Population
Male Population

| | | | | |
|---|---|---|---|---|
| Age | <30 y | 30~45 y | 45~60 y | >60 y |

β value

| | | | | |
|---|---|---|---|---|
| DE-2B | 0.247 | 0.265 | 0.679 | 0.443 |

Cp value

| | | | | |
|---|---|---|---|---|
| ACTB | 33.36 | 32.92 | 32.86 | 32.65 |

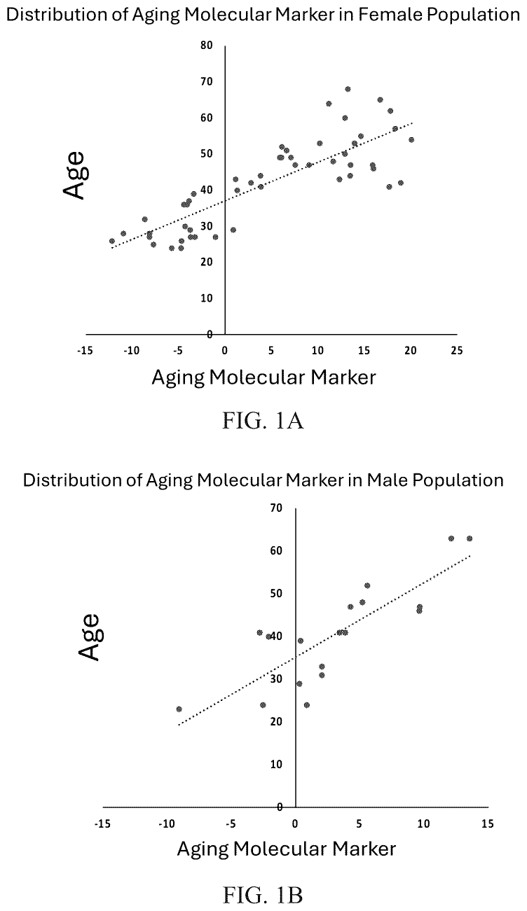

FIGS. 1A and 1B show the scatter plots for an aging molecular marker in the female and male populations, respectively. The scatter plots present data derived from two target genes, DENND2B and ACTB, measured in plasma cell-free nucleic acids from each subject. Using binomial regression analysis, we established a statistical model (ax+by+c) that reveals a significant correlation between age and the levels of nucleic acid methylation (DENND2B) and relative nucleic acid quantity (ACTB). As shown in FIGS. 1A and 1B, as age increases, the calculated values reflecting both methylation and relative nucleic acid levels also increase, further supporting a strong correlation between these two target genes and aging. Based on these results, we refer to the age-related data reflected by this model as the “aging molecular marker,” emphasizing the correlation between aging, methylation status, and relative nucleic acid quantity.

### Example 2: Comparison of Methylation Levels of Aging Molecular Marker Across Different Age Groups

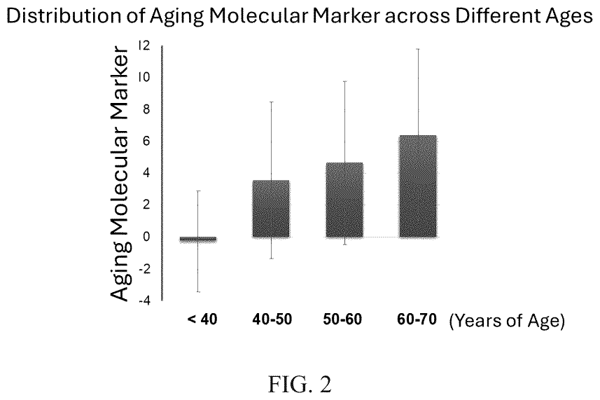

The methylation levels of DENND2B and the relative nucleic acid quantities in plasma cell-free nucleic acids were further analyzed across different age groups, so as to evaluate the progression and trend of aging by the aging molecular marker. FIG. 2 shows that the aging molecular marker increases progressively with age, indicating a significant correlation between age and both the methylation levels and the relative nucleic acid quantity in the aging molecular marker. These findings suggest that the aging molecular marker can provide valuable insights into a subject's cellular age and aging progression, serving as an important reference for evaluating physiological status.

What is claimed is:

1. A method for detecting an aging status of a subject, wherein the method comprises:
(a) extracting (i) cell-free DNA (cfDNA) from a biological sample of the subject or (ii) genomic DNA from a biological sample of the subject, wherein the cfDNA or the genomic DNA comprises a DENND2B DNA and a reference gene DNA;
(b) assaying a methylation level of one or more CpG site of the DENND2B DNA from the cfDNA or the genomic DNA;
(c) measuring a relative nucleic acid quantity of the reference gene DNA from the cfDNA or the genomic DNA; and
(d) determining an aging status of the subject based on a combination of the methylation level and the relative nucleic acid quantity.

2. The method of claim 1, wherein a pair of DENND2B methylation-specific primers used to assay the methylation level comprises SEQ ID NO: 2 and SEQ ID NO: 3, or sequences having at least 85% identity thereto.

3. The method of claim 1, wherein a DENND2B methylation-specific probe used to assay the methylation level comprises SEQ ID NO: 7, or a sequence having at least 85% identity thereto.

4. The method of claim 1, wherein the reference gene DNA comprises a ACTB DNA.

5. The method of claim 4, wherein a pair of primers used to measure the relative nucleic acid quantity comprises SEQ ID NO: 5 and SEQ ID NO: 6, or sequences having at least 85% identity thereto.

6. The method of claim 4, wherein a probe used to measure the relative nucleic acid quantity comprises SEQ ID NO: 8, or a sequence having at least 85% identity thereto.

7. The method of claim 1, wherein the determining the aging status comprises calculating an aging index by binomial regression based on the methylation level and the relative nucleic acid quantity.

8. The method of claim 1, wherein the assay of step (b) is performed using methylation specific PCR (MSP), quantitative methylation-specific PCR (QMSP), bisulfite conversion, bisulfite sequencing (BS), pyrosequencing, microarrays, or methylation DNA immunoprecipitation PCR (MeDIP-PCR).

9. The method of claim 1, wherein the biological sample comprises a liquid biopsy.

10. The method of claim 1, wherein the biological sample comprises plasma or serum.

11. A kit for detecting an aging status of a subject, comprising a pair of primers having the sequences of SEQ ID NOs: 2 and 3 and/or a probe having the sequence of SEQ ID NO: 7 for assaying methylation level of a DENND2B DNA from a biological sample of a subject.

12. The kit of claim 11, further comprising a pair of primers having the sequences of SEQ ID NOs: 5 and 6 and/or a probe having the sequence of SEQ ID NO: 8 for measuring a relative nucleic acid quantity of a reference gene DNA from the biological sample.

13. The kit of claim 12, wherein the reference gene DNA comprises a ACTB DNA.
